(19) United States
(12) Patent Application Publication
(10) Pub. No.: US 2020/0206462 A1
(43) Pub. Date: Jul. 2, 2020

(54) MEDICAL PUNCTURE INSTRUMENT

(71) Applicant: Anton HÄRLE, Ludwigsburg (DE)

(72) Inventors: Anton HÄRLE, Ludwigsburg (DE); Götz GÄRLE, Hamburg (DE)

(21) Appl. No.: 16/620,888

(22) PCT Filed: Jun. 12, 2018
(86) PCT No.: PCT/DE2018/000188

§ 371 Date: Dec. 10, 2019

(30) Foreign Application Priority Data
Jun. 14, 2017 (DE) 10 2017 005 569.6

(51) Int. Cl. A61M 25/01; A61M 25/06

(52) U.S. Cl. CPC A61M 25/0111; A61M 25/0097; A61M 25/065; A61M 25/0113

(57) ABSTRACT

The invention concerns a medical puncture instrument for the relocation of drains and catheters, in order to initiate and/or channel the fluid in the body, whereby its components are already assembled, so that an immediate usage capable instrument is delivered in a sterile packaging. The puncture instrument consists at least of a puncture tube, a handling medium for maneuvering of puncture tube and a longitudinal movable cylindrical body enclosed in the puncture instrument. In a further arrangement, the puncture instrument consists of a lock chamber for the introducing or rather the suction of the fluid. For the fixation of the cylindrical body in a desired position in the puncture instrument, a braking element can be incorporated. The handling element is open in a section, so that the cylindrical body here lies free and can be suspended.

The present invention deals with a medical puncture instrument for the insertion of drains, guide wires and catheters, in order to introduce in or remove fluids from the body. A first task comprises, to provide means to the doctor for this method of treatment, which does not imply the requirement, firstly to gather the many components individually from the package container, to sort them and then assemble them under sterile conditions, that is to firstly carry out non-aesculapian assembling work prior to his medical action. A further target of the invention is to avoid state of the art obligatory necessity, for the insertion of the catheter in the pipeline, which is already filled with the instillation fluid, to interrupt this pipeline, which means to open it, what represents a not irrelevant danger of bacterial contamination.

These problematic aspects are given in many medicinal treatment procedures, which come in consideration here and are exemplified in the embodiment for the regional anaesthesia. By such a procedure, through the instillation of local anaesthetics on the nerve meshwork or on a big nerve, that sensitizes that part of the body, this body area is made to be free of pain or anaesthetic. The instillation of the local anaesthetics to the nerval structures requires a high space oriented precision in connection with the anatomic target structures, since often success or failure is conditional by a few millimetres, or rather undesired side effects, even of serious nature, for example nerve lesions, or a respiratory paralysis can be triggered. Lately the puncture is carried out increasingly under ultrasound control and the instillation of local anaesthetics is performed with a catheter introduced through the puncture cannula and directed on the nerval structure, in doing so the catheter after the surgical procedure even serves for the post-operative pain relief and can thus stay over several days.

In the present practice, up to 10 individual components available in the packaging set are gathered under sterile conditions by the anaesthetist, sorted and have to be composed just as well in many work steps correctly and in the appropriate sequence; an unintentionally not removed sealing cap can eventually cause an interruption of the puncture procedure. A Certain problem is also represented by the threading of the mostly only 0.8 mm or lesser in diameter measuring catheter in the cannula opening or into the handling tool in case of increasingly reduced tactile performance due to the donned rubber gloves.

In case of the cost efficiency to be considered in the health care, it is also to be considered, that in the modern operation tract the operational usage cost per minute are estimated to be around 20 USD, what especially with regard to the time required for the assembling of the individual components is important. Therefore, it is striven for the introduction of a catheter in the regional anaesthesia, to reduce the number of involved people and all steps of the procedure are to be carried out possibly only through one sterile dressed person, namely the anaesthesist, alone. It does not deal only with time consuming assembling requirement and hence resulting operational cost, but also with the reliability of the procedural process under stress, in which the patient and the doctor in this situation stand, thus to possibly avoid undesired side effects like malposition of the catheter or catheter infection, which does not happen infrequently. So a current study of 3,491 regional anaesthesia procedures documented a complication rate of 12.1% (Anaesthesist volume 60, 1014; 2011).

Is the advancing of the puncture cannula carried out under the ultrasound control, the one hand managing the ultrasound head possibly till up to the catheter fixation on the skin entry site, should guarantee the sonographic position control of the distal end of the cannula and and the catheter. It is therefore desired, that all the steps of the procedure beginning with the skin puncture, the deposition of the local anaesthetic depot around the nerval structure, the advancing in of the catheter, the complete withdrawing of the puncture cannula over the catheter till up to the fixation of the catheter in the area of the skin entry can be done one-handed by the other hand. Is for the assembling or the dissembling of the different components, the interaction of one hand necessary, which manages the sonic head, the sonic head must be laid down. The retrieval of the anatomic target structure with the again gripped sonic head is difficult and time-consuming. Here it is also to be considered, that in case of the 18 up to cm long cannula the smallest changes of position on the cannula base leads to significant changes in position of the tip of the cannula in the tissue and thus injuries of important tissue structures, like for example nerves, vessels, lungs etc are can be caused.

Has the tip of the puncture cannula reached its target place and here a certain quantity of local anaesthetic is placed there as a depot for the tissue dissection, then normally the syringe, or the connecting cannula has to be disconnected from the cannula, in order to insert the catheter, which means the earlier closed and fluid wetted pipeline is now opened, what represents a further problem with regard to the sterility. For the disconnecting of the syringe or the connecting cannula from the puncture cannula both the hands must interact, and a change of hand for holding of the puncture cannula must be performed. From the local anaesthetic depot in the tissue, which stands under overpressure, drops can leak out of the proximal end of the cannula and thus the danger of the bacterial contamination for the internal side of the pipeline increases. By pushing forward of the catheter in the puncture cannula, a further fluid quantity of approximately 0.1 ml is retrogradely ejected. The inserting of the catheter though the puncture cannula should take place of course again under sonographic control, in order to ensure, that the distal end of the catheter touches on the aimed anatomic space, when it comes out from the cannula tip. For this procedural step, the one hand used for the disconnection of the syringe firstly will again take the ultra sound head and find the nerval target structure, what represents a not an easy, rather a rather time-consuming procedure.

Is the final placing of the distal end of the catheter performed, and then the puncture cannula should possibly even under the ultrasound control be withdrawn over the inserted catheter, whereby the distal catheter end isn't allowed any change of its position. The unthreading of the catheter from the puncture cannula is difficult without the use of both the hands, whereby the undesired displacement of the distal end of the catheter can occur. After the separation of the catheter and the puncture cannula, the proximal end of the catheter is introduced in a catheter coupling, with which the connecting hose is connected on the proximal end of the pipeline with the bacteria filter.

Is now or later the connecting hose is connected to a pain pump, in order to continuously instil the local anaesthetic, the volume of the air in the proximal section of the catheter, the catheter coupling and of the bacteria filter, which amounts to around 1.2 ml, is driven out from the pipeline in the perineural space, what is not only to be seen on the background of the number of airborne germs present, but air should be part in the tissue and disturbs the sonographic image. Besides it is to be considered, that the mostly less than 1 mm thick and up to 50 cm long catheter is difficult to handle with gloved fingers, caused by of reduced tactile agility on one hand, on the other hand it is even not so easy to control the elongated catheter inside the restricted area of the sterile covering.

To these procedural but also hygienic requirements the presently from the industry supplied sets correspond only insufficiently, still represents mostly only construction sets, whereby then the anaesthesist under difficult conditions and pressure of time carries out the assembling or the disassembling of up to 10 different parts in as many procedural steps.

The multi-part task of the invention is accomplished on the basis of the present invention corresponding the claims, description and images. Firstly, for the improvement of the identifiability of the puncture means in the ultrasound image, its shaft (1) should have at least in its distal part an outer surface, which increases the echogenity. This can be achieved through outer surface structures, which reflects the ultrasound in a dispersed manner or through a coating, which causes the same effect.

The next subtask, which consists of, to relieve the doctor from the assembling work using state of the art assembly sets of up to 10 different components in case for the regional anaesthesia and for the insertion of the vessel catheters, can be accomplished that way, that in place of the several components present in a set, a completely assembled puncture instrument is provided in the packaging container.

For this purpose, the so far individual components delivered as set for the regional anaesthesia are assembled already at the manufacturing site, and supplied for an immediate usage capable as a puncture instrument, which under avoidance of the above represented complication procedure is delivered in the packaging container. The gathering of such a puncture instrument from the sterile packaging is now possible with one handgrip. Similarly, it is avoided, that the various components, especially protruding and till up to 50 cm long equipment parts in the restricted anaesthesiologic operation area lies about with danger to crash, or while being gathered, jump out from the packaging sachet and are thus eventually be contaminated; even through this an erroneous assembling, for example through incorrect sequence of the procedural steps is excluded.

The explanation of the above described according to the present invention teachings serves for the following drawings.

For easy understanding of the following description, few of the here used terms are to be explained.

A conventional cannula consists of an elongated shaft, on whose distal end a puncture tip is formed. On the other, which means the proximal end, the shaft is included in a cannula frame made of plastic, whereby this process is carried out through crimping or bonding. This frame of the shaft expands towards proximal in a cannula connection part, which in the form of a plug connection or presently the mostly applied Luer-Lock-connection can be coupled with a syringe.

In this application script, the section of the cannula connection part connecting towards the proximal is due to its increasing restructuring and expanding of its function, it is also described as handling element.

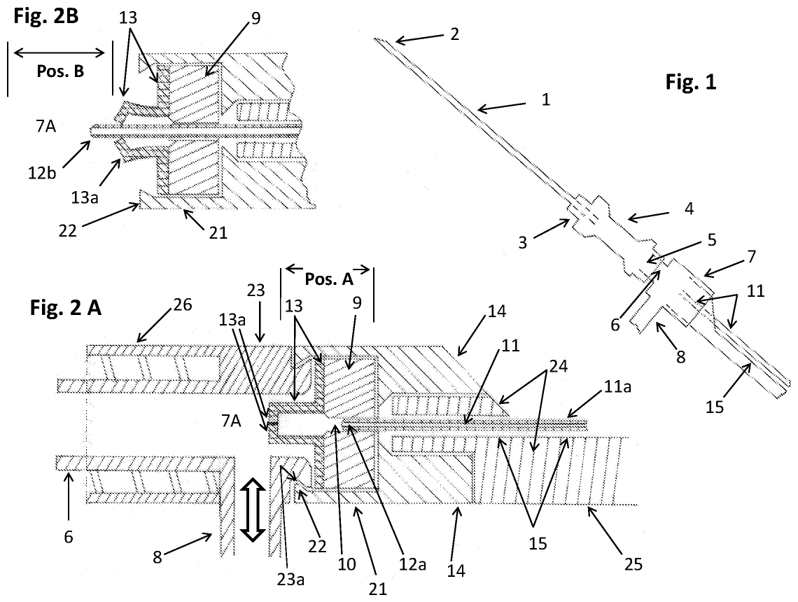

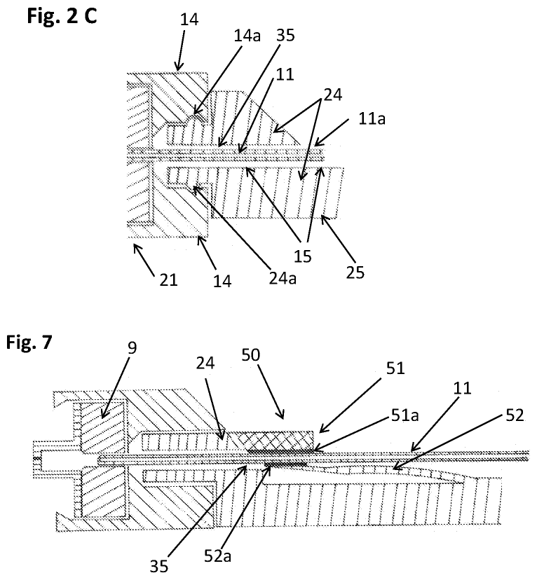

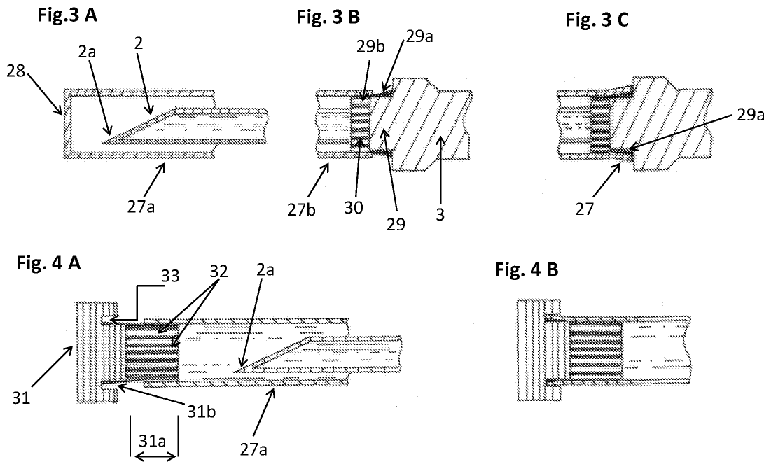

In FIG. 1 the distal section of a puncture instrument is represented in a side view. The cannula contains a cannula shaft 1, which ends distal in a cannula tip 2. On its proximal end, the cannula shaft is embedded in a cannula frame 3. Towards proximal, the cannula frame can extend in connection with a cannula connection part in a handle 4 in the form of a grip-body with an all—around formed, in cross section quadratic depression, in order to grasp the puncture instrument with two or three fingers and in case of puncture, it can be directed in a targeted manner; then it follows toward proximal the cannula connection 5 with a connector part, which is associated with a congruent connector extension 6, which is connected with an interface 7,—in the following mentioned as sluice or rather called the catheter sluice. This interface 7 is represented in FIG. 2A partially and enlarged in a longitudinal section and can be formed as catheter sluice in form of a T or Y-piece, whereby the inflow- or rather the outflow-nozzle 8 ends in a connector part, on which an extension hose is connected or can be formed, used for the instillation of the local anaesthetic. In the proximal half of the catheter sluice a rubber-elastic body 9 is located, this body is enforced through a channel 10, whose inner diameter is slightly smaller than the outer diameter of the catheter 11 and so it is measured, that there exists a sufficient, but surmountable friction strength in case of manual, axial displacement of the catheter. The thus formed catheter-brake should obstruct an undesired change of position of the catheter in the puncture instrument and thereby fix the distal end of the catheter 12a or rather 12b at the desired position in the puncture instrument, but an intentional displacement of the catheter till over the tip of the cannula is to be allowed. These requirements are fulfilled, if the friction strength of the catheter-brake is kept in the range of 0.15 to 0.6 N. Before beginning of puncture, the puncture instrument should be ventilated. This is done through spraying of instillation solution (see thick bidirectional arrow) by means of the connecting hose via the nozzle 8 in the chamber 7A of the catheter sluice; through which firstly the air found in the cannula area (1,3,4,5,7A) is driven out through the tip of the cannula 2 and the puncture instrument is ventilated completely in its distal area. For this procedure, the direction of the flow to the tip of the cannula is guaranteed and a displacement of the air found in the sluice chamber 7A is obstructed retrogradely into the catheter 11 through its tip of the catheter 12a, when distal of the elastic body 9 a technological known lip valve 13, like it is represented for example in FIG. 3 of U.S. Pat. No. 4,960,412, is installed. This lip valve 13 can be an isolated component, but can be also connected with the rubber-elastic body 9 of the catheter-brake. With this configuration, the tip of the catheter 12a lies in the predetermined position Pos A, proximal of lip 13a of the lip valve 13. Is the chamber 7a of the catheter-sluice totally void of air and filled with instillation solution, the catheter can, in the area of working surface 15 and the free lying section 11a be pushed manually towards distal, so that its tip 12b after passing of the lip valve in the chamber 7A is immersed in the instillation solution found there and the position B is reached, like represented in extracts in FIG. 2B. Is now again under pressure, the instillation solution is sprayed over the connecting hose and through the nozzle 8, this will penetrate in the tip of the catheter 12b and even the air found in the catheter is driven out retrogradely, so that now the puncture instrument if fully free of air. In order to ensure the driving out of the air from the catheter 11, the outflow of the instillation fluid via the tip of the cannula 2 is to be temporarily blocked. This can be achieved for example through modifications of the cannula protecting cover either on the proximal or distal end, which is represented in FIG. 3 and FIG. 4. For the retrograde filling of the catheter with instillation solution, the lip valve should even then ensure a water tight closure, if the catheter tip 12b is positioned in the position B. This is achieved, if the length of the slit S of the lip valve stands in a certain relation to the diameter D of the catheter, this ratio can be calculated from the equation S<(3,1415×D)/2 for each catheter. This sealing can be managed through the elastic body alone or in collaboration with the lip valve.

The proximal section of the sluice chamber 7A is built by a cap/cover 21, which intervenes distal with a protrusion 22 in a groove 23a of the distal half 23, through which the proximal and distal section of the catheter sluice are coupled preferably air- and water tight with one other. An elastic body 9 is embedded in the inner space of the cap/cover 21. The cap/cover 21 extends towards proximal in a connector part 14, in which a congruent plug connection element 24 of the handling element 25 intervenes. The distal section of the catheter sluice ends in a connector element 26, for example it is represented in form of a screw thread or a plug connection with inner lying combination process 6. In a special arrangement, both the lock sections can be arranged in the area of their connection (22, 23) in a twistable manner against each other, whereby there exists a fluid density. The distal section 23 of the sluice 7 and the cannula frame can also be formed as a single piece.

In FIG. 2c a further arrangement is represented, by which the handling element can be twisted towards the sluice cap 14. Thus one can twist the tip of the cannula with regard to the handling element This has the advantage, if the orientation of the needle tip should be adjusted individually, in order to conduct the efflux opening of the cannula in the body, especially in case of Tuohy- or side hole—needles. Hereby the connector part 14 consists of a groove 14a in which a bulge 24a of the plug connector element 24 intervenes.

The FIG. 3 A-C is longitudinal sections through a cannula protection cover 27, in which the puncture instrument is delivered in an enclosed manner FIG. 3A shows the distal end of a cannula protection cover 27a; the tip of the cannula 2 lies free in the distal end area of the cannula protection cover, which hides the sharp tip 2a of the cannula end, in order to obstruct during the preparation phase before the beginning of puncture, the injuries of the patient, or the working anaesthesist, but also to protect the slightly perforable sterile covering. On the distal end 28 the cannula protection cover is sealed water- and air tight in this arrangement.

FIG. 3B shows the proximal end 27b of the cannula protection cover, which in the delivery condition is suspended in a clamping manner over the distal section 29 of the cannula frame 3. This section of the cannula frame 3 has a distal section 29b, which on its outer surface towards outside has prominent axial passing racks 30, or rather has between these grooves.

Is the cannula protection cover only suspended in a clamping manner over this area, then in case of excess pressure existing in the cannula protection cover with regard to the surrounding, a pressure balance must take place via the grooves passing between the racks, which means air or fluid can go out. Is the cannula protection cover even suspended over the proximal section 29a of the clamping area of the cannula frame 3, it comes on the other hand to a sealing, since this area is formed without grooves, has a circular clamping area.

For improvement of the clamping effect, the proximal, corresponding area of the cannula frame has a outer diameter increasing towards proximal, like it is demonstrated in FIG. 3 C. The arrangement in form of a conical section of the cannula frame lets the clamping effect between the cannula protection cover and cannula frame increase to the extent, that firstly even a sufficient excess pressure for the retrograde filling of the end 12b of the catheter lying in the sluice chamber 7a with the instillation solution and simultaneous displacement and expulsion of the air lying in the catheter is resisted. The cannula frame 3 and the distal section 23 of the sluice 7, as well as the proximal section (lock cap) 21 and the handling element 25 can respectively be formed in single piece.

One other arrangement of the closure mechanism is represented in the longitudinal section images in the FIGS. 4A and 4B of the distal cannula protection cap 27a. FIG. 4A shows the delivery condition in the packaging, in case of which in the distal opening of the cannula protection cover 27a from distal a sealing plug 31 is slid in, which on its proximal section 31a has on the outer surface axial passing racks (32), or rather has the grooves lying in between, whereby the racks unfold a clamping effect with the cannula protection cover 27a and so the sharp cannula tip 2a is excluded from each outer contact. Through the grooves lying between the racks, there are free spaces, through which the air and/or water can come out, if in the inner part of the cannula and consequently even in the Cannula protection cover an excess pressure with regard to the surrounding is established. The withdrawal of the air and/or water can be obstructed, if the distal section 31 b of the sealing plug, which has no groove on its upper surface is impressed in the cylindrical lumen of the cannula protection cover, like it is demonstrated in FIG. 4B. In this positional relationship of the sealing plug 31 and distal end of the cannula protection cover can be finally air- and water tight. The sealing plug 31 can even have a recess 33 on the distal end of the conical thickening, in which for the improvement of the sealing the distal end of the cannula protection cover can be slid in.

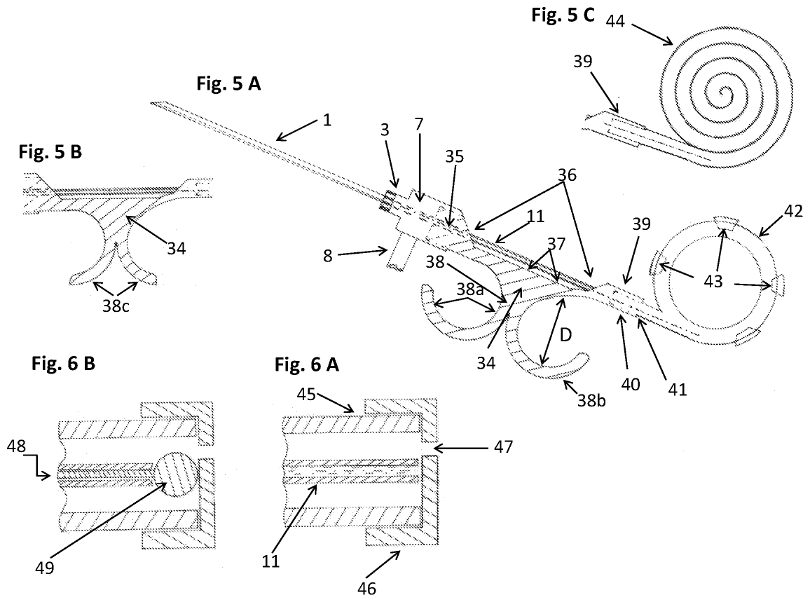

In FIG. 5A a side view of the preferable arrangement of the puncture instrument is represented, in which the handling element is arranged proximal of the catheter sluice 7 and is modified for a multi-functional handling element 34.

This handling element has a channel 35 for the catheter 11, whereby this channel is opened concentric on the upper side in a length of approximately 4 cm, and through this the catheter lies in open in this area 36. The material reduction of the handling element in this area 36 can be carried out to an extent, that the catheter lies completely free. This area 36 on the upper side of the handling element forms a contact surface 37 for a finger, preferably of thumb for axial shifting of the catheter lying here (or rather a guide or Seldinger wire) 11, whereby this is pressed with the finger against the attack (contact??) surface and thus can be displaced. On the lower side of the handling element 34 a grip arrangement 38 in form of two handle section 38a and 38b is formed, in which two fingers of a hand can be engaged and so the puncture instrument can be safely fixed and oriented in the space without using the thumb. The handle sections 38a and 38b can separately or in the area, in which they are in contact, be formed as single piece. Through this the thumb is free for other activities. If the handles 38a and 38b in the lower area, which lies between both the arrows, is somewhat elastic, it can be adjusted to the respective size of the finger, whereby in the delivery condition, it has a diameter D of approximately 12 mm. In a simplified execution, the handling element can have on the lower side instead of both round handles even only one approximately 15 to 20 mm long biconcave grip-extension 38c, which can be clamped between two fingers, like it is represented in FIG. 5 B. This grip arrangement on the lower side therefore allows by means of use of two fingers of a hand, to push forward the puncture instrument without the use of the thumb in an exact targeted manner onto the targeted anatomic spatial point. The catheter channel 35 is indicated dashed (---) in the FIGS. 1 and 5 for easy understanding, while a dash-point-dash-formation (-.-.) marks the catheter 11 in a not visible area.

Towards proximal, the handling element ends in a cylindrical section 39, on which by means of a plug connection 40 a catheter container can be connected, whose connector part 41 intervenes in the plug connection 40. The catheter container should be so formed, that through this the handling of the puncture instrument is possibly not obstructed. This assumes that in the spatial proportions it is shaped relatively small and in no level, it measures in diameter over 6 cm. For avoiding of obstruction in the puncture procedure, it is itself advantageous, if the catheter container in the plug connection 40 can be easily adjusted/determined in the various rotation positions with regard to the orientation of the handles 38a and 38b.

For better usability, the catheter container can for example consist of a helix of many on each other lying spiral windings 42, which includes the proximal part of catheter, which is not slid-in in the puncture instrument. The entire length of the container windings is oriented on the catheter length. In the side view in FIG. 5 A only the first winding of the catheter container is represented. By means of the fixing clips 43, beneath the first winding passing further spiral windings are fixed to each other; they can also be connected with one other in an interlocked manner There are other arrangements of the catheter container in consideration, which can be connected via the connector device 40/41 on the puncture instrument. In FIG. 5 c the catheter-container has for example the form of an archimedic spiral 44, whereby here the windings lie in one level, what enables a compact construction measure.

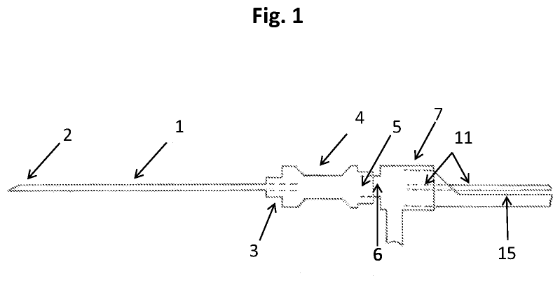

In the FIG. 6A is a longitudinal image through this, which represents the plug connection on the opposite end 45 of the catheter-container, which is sealed on this end and a retrograde sliding back of the enclosed catheter is obstructed. The sealing can even be achieved firstly secondary through a closing element 46 closing the proximal end 45. Through this before the closing of the catheter-container the inserting of the catheter from the proximal end of the catheter container is possible into the puncture instrument. On this end of the catheter—container a ventilation hole 47 is fixed in such a manner, that although air and water but not the catheter can exit through it.

FIG. 6B is likewise a longitudinal section image through which the opposite end 45 of the catheter container is represented. In the catheter 11 a core precedes 48 in form of a thin steel wire, which seals the catheter lumen to a large extent, but can be pulled out from the catheter. On the proximal end of this core 48 a blocking element 49 is arranged with a cross section, which is larger than that of inner diameter of the catheter and is represented here as a ball.

When the catheter is shifted with the thumb on the attack surface 37 towards distal to the tip of the cannula and finally even left out from this, the catheter glides out of the catheter—container successively.

Instead of the represented catheter-container, even another container holding the proximal part of the catheter can be connected on the plug connection of the cylinder section 39, for example in form of a foil bag or any other form.

In case of arranging of the catheter according to FIG. 6B, which means with one of the core 48 sealing lumen, the distal catheter tip 12 a can already be pushed forward in the position Pos. B. Through this the venting procedure of the puncture instrument can be simplified. Now only the expelling of the gases found in the connecting hose, sluice chamber and cannula area is required through the spraying of the instillation fluid; the success of this measure is recognized by the emergence of water/fluid on the distal end of the cannula protection cap.

When the puncture procedure is concluded, the puncture instrument is slowly withdrawn over the catheter towards proximal, whereby simultaneously in the area of the attack surface 37 the catheter in the same amount it is pushed forward with regard to the puncture instrument. The puncture instrument can then be removed over the catheter end which is gliding out of the catheter—container. On the proximal catheter end then a catheter coupling is then fixed, in whose proximal connector connection a bacteria filter can be slid in/connected.

In FIG. 7 a further arrangement of a braking element 50/52 for the cylindrical body 11 is represented, which is arranged in the area of the handling element 25 and is connected proximal on the plug connection element 24. It consists on the one hand of an extension 51 of the upper half of the plug connection element 24, on the other hand of a flexible—elastic lamella 52, which originates proximal from the attack surface 15 withdrawing towards distal from this, laying out on the lower side of the catheter and presses the catheter/cylindrical body towards above against the extension 51. In the contact area with the the extension 51 and/or the flaps 52 has a rubber coating 51a or rather 52a, in order to achieve the braking effect. This contact area can be arranged flat and/or corresponding the outer contour of the cylindrical body 11 be round/or congruent. The pressing force of lamella 52 is so arranged, that a friction force of 0.15 till 0.5 N exists. Is for the shifting of the cylindrical body 11 pressure is applied on this from above, finally the lamella is pressed down and through this it removes the contact of the cylindrical body with the rubber coated parts 51a and/or 52a of the braking element. The braking element 9 and 50 can be so coordinated with one other, that both exclude a shifting of the cylindrical body to a large extent; on the other hand each can alone give rise to a desired braking effect. In the puncture instrument can also however only one of both can be developed.

Summarizing one can describe the handling of the puncture instrument like the following. All part components of the puncture instrument are present already connected by the manufacturer in the sterile packaging and are without any assembling requirement immediately ready to use. The puncture instrument also consists of a braking element, which holds the cylindrical body at a certain spot, but an arbitrary shifting is possible. The entire puncture instrument can be grasped by means of a grip form on the lower side of the handling element with one hand and be removed with a hand grip from the sterile packaging. On the free end of the extension hose is now, either a bacteria filter, as far as one such is used-, or a syringe filled with a local anaesthetic is directly fixed. Then the local anaesthetic is sprayed as long into the system, till all the air is driven out of the puncture instrument and the fluid comes out from the needle tip. Then the anaesthesist looks for with the hand by means of sterile packaged ultra sound head the interesting nerval structure and brings with the other hand the puncture instrument on the suitable skin puncture and subsequently to targeted anatomical spatial place. Is the anatomic desired space point reached with the tip of the puncture instrument, then the catheter present in the puncture instrument is pushed forward at the desired anatomic place.

The submitted invention of a puncture instrument can naturally be used advantageously in other medical procedures with corresponding adjustments.

1. Puncture instrument for medicinal use for the insertion of long stretched cylindrical means in the body, which consists of many components at least of a puncture aid with a metallic shaft (1), which ends on its distal end in a cannula tip (2) and its proximal end in a cannula frame (3), a handling-medium (4,34) for targeted advancing of of the puncture instrument onto a certain anatomic space point in the body and a long stretched cylindrical means enclosed in the puncture aid, being axially movable (11), thus characterized by, that these components are present as a complete mounted and ready for immediately be used puncture instrument in sterile packaging, the distal end (12a or 12b) of the cylindrical formed means is placed at a pre-determined position in the puncture instrument, the handling element (4; 36; 38) is formed as a grip body with a grip capable upper surface arrangement for finger and is connected with a cannula frame (5), and the cylindrical formed means, which is present in a channel, formed by a the shaft and the handling element, has an essentially large length than the surrounding channel, the cylinder forming aid (11) being movable as regard to the shaft (1) and the handling element and protruding proximal from the handling area.

2. Medicinal puncture instrument according to claim 1, thus characterized by, that the puncture aid at least has in its distal area for better recognizability in the ultrasound image an outer upper surface, which increases the echogenity.

3. (canceled)

4. A sluice chamber element according to claim 31, thus characterized by, that the cylinder forming aid (11) is a catheter.

5. A sluice chamber element according to claim 31, thus characterized by, that the cylinder forming aid (11) is a solid body.

6. A sluice chamber element according to claim 31 thus characterizes by, that the cylindrical body is a steel wire.

7. A sluice chamber element according to claim 31, thus characterized by, that the process (8) is already connected with an extension hose.

8. A sluice chamber element according to claim 31, thus characterized by, that inside that sluice chamber a brake element (9; 13) is established, which retains the long stretched cylindrical body (11) in the desired slot position.

9. A sluice chamber element according to claim 31, thus characterized by, that the friction force of the brake is kept in the range of 0.15 till 0.6 N.

10. A sluice chamber element according to claim 31, thus characterized by, that despite the brake effect the cylindrical body can be deliberately pushed axially.

11. (canceled)

12. (canceled)

13. Medicinal puncture instrument according to claim 1, thus characterized by, that the brake element is established in the area of the handling area (36) and is located proximal of the proximal opening of the channel (35) for the cylindrical body (11).

14. Medicinal puncture instrument according to claim 1, thus characterized by, that the brake elements (9,13,50) can be combined.

15. Medicinal puncture instrument according to claim 1, thus characterized by, that the brake (50; 52) is localized) in the handling medium (34) the braking effect can be modified one-handed.

16. Medicinal puncture instrument according to claim 1, thus characterized by, that a device for the twisting of the shaft or rather the needle tip (2) with regard to the handling element (34).

17. Medicinal puncture instrument according to claim 1, thus characterized by, that the device for the change of the spatial orientation of the needle tip in the area of the lock (7) is localized and can be achieved with single hand.

18. (canceled)

19. A sluice chamber element according to claim 31 thus characterizes by, that that the elastic body (9) has a fluid sealing function in its channel area (10).

20. Medicinal puncture instrument according to claim 1, thus characterized by, that the cannula protection cover (27) can have two positions with regard to the cannula frame (3), which guarantees in one position the impermeability of the air and in the other position the impermeability of the air.

21. Medicinal puncture instrument according to claim 1, thus characterized by, that on the distal end of the cannula protection cover (27) a plug (31) is localized, which can have two positions, which guarantees in one position the permeability of the air and in the other position the impermeability of the air.

22. Handling element (34) as part of a medical puncture aid is formed with grip devices (38) and so the maneuvering of the puncture instrument is enabled with two fingers without the help of the thumb.

23. Handling element according claim 22, thus characterized by, that the elastic grip devices (38) of the handling element (34) has an adaptation capability to the size of the intervening finger.

24. Handling element according to claim 1, thus characterized by, that on the puncture instrument in the delivery condition a container is attached for inclusion of a cylindrical body (11) protruding towards proximal side.

25. Handling element according to claim 1, thus characterized by, that the container is rotatable with regard to the handling element.

26. Handling element according to claim 1, thus characterized by, that the container of the cylindrical body (11) lies in its largest extent in spatial section below 8 cm.

27. Handling element according to claim 1, thus characterized by, that the container for of the cylindrical body (11) lies in its largest extent in a spatial section below 5 cm.

28. Handling element according to claim 1, thus characterized by, that the container is formed from spiral windings (44) in a spatial section.

29. Handling element according to claim 1, thus characterized by, that spiral windings lies helix type in various spatial sections (42).

30. Handling element according to claim 1, thus characterized by, that the container consists of for example sachet, flaps or other forms deviating from a spiral winding.

31. A sluice chamber element (7) for use in a medical puncture aid, positioned in between the cannula base (5) and the handling element (24), said sluice chamber element providing an axial passage through proximal and distal openings for a long stretched cylindrical body (11) and exhibiting laterally a third opening with a process (8) for passage of fluids.
